(19) United States
(12) Patent Application Publication
(10) Pub. No.: US 2014/0276184 A1
(43) Pub. Date: Sep. 18, 2014

(54) METHOD AND SYSTEM FOR TMS DOSE ASSESSMENT AND SEIZURE DETECTION

(71) Applicant: NEXSTIM, Helsinki (FI)

(72) Inventor: Tuomas Neuvonen, Espoo (FI)

(73) Assignee: NEXSTIM, Helsinki (FI)

(21) Appl. No.: 13/833,954

(22) Filed: Mar. 15, 2013

(51) Int. Cl. A61B 5/0484; A61B 5/00

(52) U.S. Cl. CPC A61B 5/0484; A61B 5/4094
USPC 600/544

(57) ABSTRACT

A method of and system for monitoring a patient's EEG (electroencephalogram) during TMS (Transcranial Magnetic Stimulation) are disclosed. The system comprises a TMS device to generate, when in an active state, a plurality of magnetic pulses, which can be applied either as a burst, comprising a plurality of pulses grouped together, or as individual pulses, to the patient's head, in accordance with a TMS treatment protocol. An EEG system is provided to measure EEG data resulting from the TMS treatment protocol being applied to the patient. The system further comprises control means in communication with the TMD device and the EEG system, the control means being arranged to activate the EEG system during the time periods when the TMS device is not generating pulses, such that the EEG data measurement is continuously applied or interleaved with the magnetic pulses being generated in accordance with the TMS treatment protocol, so as to monitor treatment efficacy and detect potential seizures.

### FIELD OF THE INVENTION

This invention relates to a method and device for TMS dose assessment and seizure detection.

### BACKGROUND TO THE INVENTION

Transcranial magnetic stimulation (TMS) is a technique for stimulating the human brain noninvasively. In particular, TMS causes depolarization or hyperpolarization in the neurons of the brain. TMS uses electromagnetic induction to induce weak electric currents using a rapidly changing magnetic field; this can cause activity in specific or general parts of the brain with minimal discomfort, allowing the functioning and interconnections of the brain to be studied. TMS thus uses the principle of inductance to get electrical energy across the scalp and skull without the pain of direct percutaneous electrical stimulation. It involves placing a coil of wire on the scalp and passing a powerful and rapidly changing current through it. This produces a magnetic field which passes unimpeded and relatively painlessly through the tissues of the head. This magnetic field, in turn, induces a much weaker electrical current in the brain. In order to induce enough current to depolarize neurons in the brain, the current passed through the stimulating coil must start and stop or reverse its direction within a few hundred microseconds.

TMS is currently used in several different forms. In a first form, called single-pulse TMS, a single pulse of magnetic energy is delivered from the coil to the patient. In another form, namely repetitive TMS (rTMS), a train of pulses is delivered over a particular time period, with various frequency patterns. The frequency sequences upregulate the cortical excitability and in some diseases (e.g. depression) the use of such patterns is advantageous. However, high frequency patterns carry a risk of elevated seizure risk. Safety limits for stimulation intensity and frequency are described in international consensus papers (eg. Rossi et al 2009, Wassermann et al 1996).

In order to monitor the safety and efficacy of a TMS application, one known way would be to monitor the patient's status visually, during and after the TMS application. This subjective assessment for safety purposes is thus available, and is typically based on a questionnaire. However, online feedback for the effectiveness of the treatment is missing, and this protocol is unable to detect a seizure in time.

A further known way of monitoring the safety and efficacy of a TMS application is to monitor a patient's EEG before and after a TMS session.

An electroencephalogram (EEG) is a record of specific brain wave patterns in a patient. EEG systems permit the recording of the brain wave patterns. An EEG system typically includes a plurality of conductive electrodes that are placed on a patient's scalp. These electrodes are typically metal and are connected to a preamplifier that processes the signals detected by the electrodes and provides amplified signals to an EEG machine. The EEG machine contains hardware and software that interprets the signals to provide a visual display of the brain wave activity detected by the electrodes. This brain wave activity is typically displayed on a strip chart recorder or computer monitor.

In practice, the use of an EEG involves measuring an evoked response before and after a TMS session, and then measuring the spontaneous EEG or non-magnetically evoked EEG responses after the treatment session. However, applying an EEG measurement after the TMS treatment session could prolong the entire treatment session significantly.

Yet a further known way of monitoring the safety and efficacy of a TMS application is to monitor a patient's EEG during a TMS session. However, monitoring a patient's EEG during a TMS pulse presents technical problems, since TMS-compatible EEG systems can generally not accommodate TMS measurements because the high-energy dynamic magnetic fields generated by the TMS device induces undesirable voltages in the EEG leads, thereby making the use of conventional EEG hardware unsuitable for the safe and effective monitoring of TMS therapy. In particular, at least the preamplifiers used in current EEG systems experience saturation caused by the magnetic field generated by the TMS system. Since the electrodes used to monitor the EEG are typically in close proximity to the TMS coil, the magnetic pulse induces a signal in one or more of the EEG electrodes which causes the EEG preamplifiers to saturate. Typical preamplifiers used in EEG systems take a relatively long time to recover after being saturated by a TMS pulse.

One known way of monitoring EEG during TMS includes amplifiers in the EEG system that use a sample-and-hold circuit to pin the amplifier to a constant level during the TMS pulse. The amplifiers are said to recover within 100 microseconds after the end of the TMS pulse. Although this system appears to allow monitoring of the EEG within a short time after the end of a TMS pulse, additional gating and synchronizing circuitry is necessary to control the operation of the EEG amplifiers with respect to the TMS system. Additional gating and sampling circuitry is undesirable because it requires additional circuitry and because it can be complicated.

An additional complication that occurs when a patient's EEG is monitored during TMS occurs because of the use of metal electrodes to sense EEG signals. Large eddy currents induced by the TMS pulse or pulses in the metal electrodes can cause localized heating that may result in burns to a patient's scalp. This presents a safety hazard.

US 2002/007128 A1 discloses yet a further way of monitoring EEG during TMS. This prior art document discloses a system and method in which there is synchronisation between the timing of operation of the TMS system and the timing of operation of the EEG system. Instead, this disclosure provides a controlling arrangement that monitors the signals provided by the EEG system during operation of the TMS system and stops operation of the TMS system if the EEG signals are in an undesirable state.

### SUMMARY OF THE INVENTION

According to a first aspect of the invention there is provided a system for monitoring a patient's EEG (electroencephalogram) during TMS (Transcranial Magnetic Stimulation), the system comprising:
a TMS device to generate, when in an active state, a plurality of magnetic pulses, which can be applied either as a burst, comprising a plurality of pulses grouped together, or as individual pulses, to the patient's head, in accordance with a TMS treatment protocol;
an EEG system to measure EEG data resulting from the TMS treatment protocol being applied to the patient; and
control means in communication with the TMS device and the EEG system, the control means being arranged to activate the EEG system during the time periods when the TMS device is not generating pulses, such that the EEG data measurement is continuously applied or interleaved with the magnetic pulses being generated in accordance with the TMS treatment protocol, so as to monitor treatment efficacy and detect potential seizures.

In an embodiment, the TMS device is arranged to generate a signal when it is not in an active state and to send this signal to the control means, with the control means accordingly being arranged to trigger the operation of the EEG system when the TMS device is not active.

In an embodiment, the system comprises storage means to store the measured EEG data.

In an embodiment, prior to the TMS device applying the plurality of pulses, the TMS device sends a preparatory signal to the EEG system, the preparatory signal being used by the control means to trigger a recorder to record the EEG data in the storage means.

In an embodiment, the system comprises a seizure monitoring module to detect or predict a seizure in the patient.

In an embodiment, the seizure monitoring module measures the spectral properties of the spontaneous oscillatory brain activity when the TMS device is not generating pulses, and to compare the resulting measurement result to an expected or desired profile so as to detect or predict a seizure in the patient.

In an embodiment, the seizure monitoring module is in communication with the control means, so that in the event of a seizure being detected or predicted, the control means can stop the operation of the TMS device.

In an embodiment, the seizure monitoring module, as with the EEG system, is arranged to be activated during the time periods when the TMS device is not generating pulses.

In an embodiment, the system comprises a dosage monitoring module to enable an operator to adjust the dosage of the pulses provided by the TMS device in a subsequent treatment protocol.

In this embodiment, the TMS device is arranged to apply a plurality of pulses during a wait period, the wait period being defined as the time period between bursts of pulses in accordance with the treatment protocol.

In an embodiment, the plurality of pulses are individual pulses generated at random intervals, with the control means being arranged to deactivate the EEG system during the transmission of these pulses and to then activate the EEG system immediately thereafter to monitor and measure the patient's response.

In an embodiment, prior to applying the plurality of individual pulses during the wait period, the TMS device sends a preparatory signal to the EEG system via the control means to enable the EEG system to activate its protection circuitry.

In an embodiment, the control means is arranged to automatically adjust the profile of the pulses being generated by the TMS device and/or recommend an adjusted profile of pulses to be generated by the TMS device in a subsequent treatment session.

In an embodiment, the TMS device comprises a capacitor, to generate an electric current and thus induce a magnetic field to provide the magnetic pulses, a coil or probe to deliver the magnetic pulses, and high voltage charging circuit to charge the capacitor.

In an embodiment, the TMS device is arranged to generate and send the signal to the control means to indicate when it is not in an active state during a time period when the TMS device is not charging the capacitor.

In an embodiment, the system comprises a navigation system to assist in the position of the TMS device.

In an embodiment, the system comprises a patient-response device (typically embedded in the EEG system) to induce visual, sensory, auditory or other types of stimulation, when the TMS device is not generating pulses, for subsequent measurement.

In an embodiment, the EEG system comprises an amplifier and protection circuitry designed to accommodate the high voltages and current associated with the TMS device, with the control means being arranged to transmit a preparatory signal to the EEG system, prior to a TMS magnetic pulse, so as to activate the protection circuitry.

According to a second aspect of the invention there is provided a method of monitoring a patient's EEG (electroencephalogram) during TMS (Transcranial Magnetic Stimulation), the method comprising:
generating a plurality of magnetic pulses, which can be applied either as a burst, comprising a plurality of pulses grouped together, or as individual pulses, to the patient's head, in accordance with a TMS treatment protocol;
measuring EEG data resulting from the TMS treatment protocol being applied to the patient, wherein the EEG data is measured during the time periods when there are no magnetic pulses being generated, such that the step of measuring EEG data is continuously applied or interleaved with the magnetic pulses being generated in accordance with the TMS treatment protocol, so as to monitor treatment efficacy and detect potential seizures.

In an embodiment, the method comprises generating a signal when no magnetic pulses are being generated, with this signal in turn triggering the measuring of the EEG data.

In an embodiment, the method comprises storing the measured EEG data.

In an embodiment, prior to the generation of the magnetic pulses, the method comprises generating a preparatory signal, with this signal in turn triggering the storing of the EEG data.

In an embodiment, the method comprises:
measuring the spectral properties of the spontaneous oscillatory brain activity when there are no magnetic pulses being generated; and
comparing the resulting measurement to an expected or desired profile so as to detect or predict a seizure in the patient.

In an embodiment, the method comprises stopping the generation of the magnetic pulses in the event of a seizure being detected or predicted.

In an embodiment, the method comprises:
applying a plurality of individual pulses at random intervals during a time period when there are no magnetic pulses being generated in accordance with the treatment protocol; and
measuring the resulting EEG data, so as to enable an operator to adjust the dosage of the TMS pulses in a subsequent treatment protocol.

In this embodiment, the method comprises:
deactivating the measuring of the EEG data during the application of the plurality of individual pulses at random intervals; and
measuring the EEG data immediately thereafter to monitor and measure the patient's response.

In this embodiment, the method comprises automatically adjusting the profile of the pulses and/or recommending an adjusted profile of pulses to be generated in a subsequent treatment session.

In an embodiment, the method comprises inducing visual, sensory, auditory or other types of stimulation, when there are no magnetic pulses being generated in accordance with the treatment protocol for subsequent EEG data measurement.

### BRIEF DESCRIPTION OF THE DRAWINGS

The invention will be described, by way of example only, with reference to the accompanying drawings in which:

FIG. 1 shows a high level schematic view of a system for monitoring a patient's EEG (electroencephalogram) during TMS (Transcranial Magnetic Stimulation), according to an embodiment of the present invention,

FIG. 2 shows a timeframe schematic of a TMS protocol and an adjacent EEG protocol, to detect a seizure in the patient, according to one aspect of the present invention,

FIG. 3 shows a timeframe schematic of a TMS protocol and an adjacent EEG protocol, for dosage monitoring, according to a further aspect of the present invention,

FIG. 4 shows a timeframe schematic of a TMS protocol and an adjacent EEG protocol, for dosage monitoring using intermediate random TMS pulses, according to yet a further aspect of the present invention, and

FIG. 5 shows a high level flowchart representing a method of monitoring a patient's EEG (electroencephalogram) during TMS (Transcranial Magnetic Stimulation), according to a further embodiment of the present invention.

### DETAILED DESCRIPTION OF PREFERRED EMBODIMENTS

Referring first to FIGS. 1 to 4, a system 10 for monitoring a patient's EEG (electroencephalogram) during TMS (Transcranial Magnetic Stimulation) is shown. The system 10 comprises a TMS device 12 to generate, when in active state, a plurality of magnetic pulses. The pulses can be applied either as a burst, comprising a plurality of pulses grouped together, as indicated by arrow 14 in FIGS. 2 to 4, or as individual pulses, as indicated by arrows 16 in FIG. 4, to the patient's head 18 in accordance with a TMS treatment protocol.

Based on established safety guidelines, TMS treatment protocols often combine active stimulation (corresponding to arrows 14 in FIGS. 2 to 4) with long pauses in between (as indicated by arrows 20) in FIGS. 2 to 4. The duration of these pauses often exceeds the duration of active stimulation, as clearly shown. The waiting periods defined by the pauses are to be used for measuring diagnostic data that could be interpreted in real-time and used for guiding the TMS treatment protocol, as will be explained in more detail further below. A typical treatment protocol used for depression treatment, using rTMS, consists of a burst of 40 pulses (arrow 14 in FIGS. 2 to 4) delivered at 10 Hz, followed by a 26 second waiting period (arrows 20 in FIGS. 2 to 4), repeated until 3000 pulses have been delivered.

The system 10 further comprises an EEG system 22 to measure spontaneous EEG data associated with the TMS treatment protocol being applied to the patient. The purpose of the EEG system 22, as will be explained in more detail further, is to monitor the patient's brain activity in order to extract diagnostic information during the treatment protocol.

The system 10 further comprises control means 24 in communication with the TMS device 12 and the EEG system 22. The control means 24 comprises a controller 26 to activate the EEG system 22 during the time periods when the TMS device 12 is not generating pulses, as indicated by blocks 28 in FIGS. 2 to 4. The EEG data measurement is thus continuously applied or interleaved with the TMS treatment, so as to monitor treatment efficacy and detect potential seizures.

In an embodiment, the TMS device 12 is arranged to generate, and send to the control means 24, a signal when it is not in an active state (i.e. corresponding to a “no-stimulation” event during a treatment protocol, i.e. arrow 20 in FIGS. 2 to 4). The control means 24 is accordingly arranged to trigger the operation of the EEG system 22 when the TMS device 12 is not active.

The system 10 may comprise storage means 30 to store the measured EEG data. In an embodiment, prior to the TMS device 12 applying the plurality of pulses, the TMS device 12 sends a preparatory signal to the EEG system 22, the preparatory signal being used by the control means 24 to trigger a recorder 32 to record the EEG data in the storage means 30.

In an embodiment, the system 10 comprises a seizure monitoring module 34 (which may be embodied within the EEG system 22) to detect or predict a seizure in the patient. The seizure monitoring module 34 measures the spectral properties, including frequency, burst suppression, and phase-locked oscillations, of the spontaneous oscillatory brain activity when the TMS device 12 is not generating pulses, and to compare the resulting measurement result to an expected or desired profile. In other words, this module 34 matches measured spectral properties to the individual's EEG properties (i.e. the patient's typical pre-seizure activity) so as to determine the effects of the operation of the TMS device 12 on the patient.

The seizure monitoring module 34 is in communication with the control means 24, so that in the event of a seizure being detected or predicted, the control means 24 can stop the operation of the TMS device 12. In this application, the EEG system 22 quantifies, and records in the storage means 30, spectral properties of the measured EEG signals, and then compares this quantified dated to a benchmark EEG. Typically, if pre-seizure activity is detected, an operator is notified to decide whether to stop treatment. If seizure activity is detected, the operator is notified to stop treatment.

In an embodiment, the seizure monitoring module 34, as with the EEG system 22, is arranged to be activated during the time periods when the TMS device 12 is not generating pulses, corresponding to blocks 20 in FIGS. 2 to 4.

In a further application, the EEG system 10 comprises an amplifier 36 to amplify bioelectric potentials associated with neuronal activity of the brain, to enable unipolar and bipolar EEG measurements, protection circuitry 38 designed to accommodate the high voltages and current associated with the TMS device, and a control unit 40 to connect and control the components of the EEG system 22. The control means 24 may be arranged to transmit a preparatory signal to the EEG system 22, prior to a TMS magnetic pulse being generated, so as to activate the protection circuitry 38, and thereby prevent amplifier saturation.

In an embodiment, the TMS device 12 comprises a capacitor 42, to generate an electric current and thus induce a magnetic field to provide the magnetic pulses, a coil or probe 44 to deliver the magnetic pulses, a high voltage charging circuit 46 to charge the capacitor 42, and a control unit 48. The capacitor 42 is charged for a few seconds, with the stored energy then being released into the coil 44 as a single or multiple pulses.

In an embodiment, the TMS device 12 is arranged to generate and send the signal to the control means 24 to indicate when it is not in an active state during a time period when the TMS device 12 is not charging the capacitor 42. This enables the EEG system 22 to operate in an environment that is free of commonly known artefacts.

Thus, in use, in one embodiment, as shown in FIGS. 2 to 4, the system 10 initiates a treatment sequence, e.g. 10 Hz/4 seconds of continuous stimulation with 26 seconds inter-burst intervals. During stimulation, as indicated above, the EEG system 22 is turned off so as to prevent amplifier saturation. After stimulation, EEG monitoring is activated in response to the TMS device 12 sending a “non-stimulation” signal to the EEG system 22, corresponding to the start of the waiting period. If the TMS capacitor 42 needs to be charged, the TMS device 12 sends a preparatory signal to the EEG system 22 to prevent recording during the charging procedure.

In a further application, with specific reference to FIGS. 1, 2 and 4, the system 10 comprises a dosage monitoring module 50, typically embodied in the EEG system 22, to enable an operator to adjust the dosage of the pulses provided by the TMS system 12 in a subsequent treatment protocol. This dosage monitoring ability may be done in one of two ways, with reference to FIGS. 3 and 4, respectively.

Referring first to FIG. 3, the TMS treatment sequence may be applied in a conventional manner, and as described above, namely 10 Hz/4 seconds of continuous stimulation, with 26 seconds inter-burst intervals to define the waiting period, with a typical treatment comprising 75 such sequences. In this application, the dosage monitoring module 50 of the EEG system 22 is arranged to facilitate the acquisition of averaged EEG epochs with a reasonable signal-to-noise ratio, and to perform quantitative monitoring (e.g. spectrum, coherence and connectivity).

Turning now to FIG. 4, the TMS treatment sequence described above may be applied, namely 10 Hz/4 seconds of continuous stimulation, with 26 seconds inter-burst intervals. In addition, in this application, the TMS device 12 is arranged to apply a plurality, e.g. 3-7, of individual pulses 16 at random intervals during the waiting period 20. As indicated above, the waiting period 20 is defined to be the time period between bursts 14 of pulses in terms of the treatment protocol.

The control means 24 is arranged to deactivate the EEG system 22 during the transmission of these pulses, as indicated by rectangular blocks 52 in FIGS. 2 to 4, and to then activate the EEG system 22 immediately thereafter, as indicated by blocks 28, to monitor and measure the patient's response.

As indicated above, prior to applying the plurality of individual pulses 16 during the waiting period 20, the TMS device 12 sends a preparatory signal to the EEG system 22 via the control means 24 to enable the EEG system 22 to activate its protection circuitry 38.

In use, after each treatment sequence, the properties of evoked responses are quantified so as to extract measures of local excitability (e.g. amplitude, latency, surface properties etc.) and global connectivity (e.g. inter-hemispheric conduction time, amplitude ratio etc.). At the end of the treatment session, the resultant data is stored in the storage means 30 for comparison or trending purposes. Based on the information recorded during the treatment session, the operator can adjust the dose if need be.

In an embodiment, the control means 24 is arranged to automatically adjust the profile of the pulses being generated by the TMS device 12 and/or recommend an adjusted profile of pulses to be generated by the TMS device 12 in a subsequent treatment session.

In an embodiment, the system 10 comprises a navigation system to assist in the accurate position of the TMS device (i.e. the positioning of the coil or probe 44).

In an embodiment, the system 10 comprises a patient-response device 54, which is typically embedded in the EEG system 22, to induce visual, sensory, auditory or other types of stimulation. This stimulation is applied when the TMS device 12 is not generating pulses, for subsequent measurement. Again, the results of the patient's responses to the induced stimulation are typically stored in the storage means 30.

Finally, with reference to FIG. 5, a method 80 of monitoring a patient's EEG during TMS will now be described. The method comprises the step of generating a plurality of magnetic pulses, as indicated by block 82, which can be applied either as a burst, comprising a plurality of pulses grouped together, or as individual pulses, to the patient's head. This may be done in accordance with a TMS treatment protocol.

The method 80 further comprises the step of measuring EEG data resulting from the TMS treatment protocol being applied to the patient, as indicated by block 84. The EEG data is measured during the time periods when there are no magnetic pulses being generated, such that the step of measuring EEG data is continuously applied or interleaved with the magnetic pulses being generated in accordance with the TMS treatment protocol, so as to monitor treatment efficacy and detect potential seizures.

The present disclosure thus provides a configuration that enables an artefact free measurement of spontaneous EEG and evoked responses during a TMS treatment protocol organised in stimulation patterns. The embedding or interleaving of EEG measurements during TMS treatment enables the personalisation and optimization of the treatment sequence.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A method of monitoring a subject's electroencephalogram (EEG) during Transcranial Magnetic Stimulation (TMS) session, the method comprising the steps of;
after a stimulation pulse from a TMS coil, sending a no stimulation signal to an EEG device,
activating the EEG device based on the no stimulation signal,
measuring spontaneous EEG signals from the subject,
comparing the spontaneous EEG signals to a predetermined EEG template,
detecting pre-seizure and/or seizure activity in the comparison.

2. A method in accordance with claim 1, further comprising the steps of;
measuring spontaneous EEG signals from a subject prior to any stimulation pulses from a TMS coil,
generating a predetermined EEG template for the subject based on the pre stimulation measured EEG signals, and
wherein said generated predetermined EEG template is used in the comparison of the post stimulation pulse measured EEG signals.

3. A method in accordance with claim 1, wherein the predetermined EEG template includes known pre-seizure activity.

4. A method in accordance with claim 1, further comprising the steps of;
prior to activating the TMS coil, sending a stimulation signal to the EEG device, and
deactivating the EEG device based on the stimulation signal.

5. A method in accordance with claim 1, further comprising the step of;
sending the no stimulation signal after a capacitor or capacitor bank of a TMS device connected to the TMS coil has finished charging after a stimulation pulse.

6. A method in accordance with claim 1, further comprising the steps of;
after sending a no stimulation signal to the EEG device, sending an additional signal to the EEG device indicating that one or more capacitors of a TMS device connected to the TMS coil are, or have finished charging after a stimulation pulse, and
activating the EEG device based on at least the no stimulation signal and the additional signal.

7. A method in accordance with claim 1, further comprising the step of;
notifying a user of the TMS coil of the presence and/or lack of presence of detected pre-seizure activity.

8. A method in accordance with claim 1, further comprising the steps of;
sending a stop signal to a TMS coil device connected to the TMS coil if pre-seizure and/or seizure activity is determined, and
preventing TMS stimulation from the TMS coil based on the stop signal.

9. A method in accordance with claim 1, further comprising the steps of;
prior to a TMS stimulation pulse, sending a stimulation signal to the EEG device, and
activating a compensation mechanism within the EEG device to prevent EEG saturations.

10. A method in accordance with claim 9, wherein said compensation mechanism is gating.

11. A method of monitoring Transcranial Magnetic Stimulation (TMS) dose build-up in a subject, said method comprising the steps of;
during a wait period after a predetermined sequence of TMS stimulation pulses, sending a preparatory signal to an EEG device,
based on the preparatory signal, initiating recording and averaging of evoked EEG responses of the subject,
prior to a subsequent predetermined sequence of TMS stimulation pulses, sending a stop signal to the EEG device,
based on the stop signal, terminate recording and averaging of evoked EEG responses of the subject,
determining local excitability and/or global connectivity based on the recorded and averaged evoked EEG responses during said wait period,
storing said determined local excitability and/or global connectivity for the wait period,
comparing said stored determined local excitability and/or global connectivity for the wait period to a determined local excitability and/or global connectivity for a previous wait period,
based on said comparison, making an adjustment to the predetermined sequence of TMS stimulations for a subsequent sequence of TMS stimulations if it is determined that an adjustment should be made.

12. A method in accordance with claim 11, wherein based on the preparatory signal, the EEG initiates a compensatory mechanism to prevent EEG saturations.

13. A method in accordance with claim 12, wherein the compensatory mechanism is gating.

14. A method in accordance with claim 11, wherein the determined local excitability includes local amplitude(s), latency, surface properties or a combination thereof.

15. A method in accordance with claim 11, wherein the determined global connectivity includes global conduction time, interelectrode comparison of amplitude, latency or a combination thereof.

16. A method in accordance with claim 11, further comprising the steps of;
after a plurality of wait periods, determining extract measurements of local excitability and/or global connectivity, and
making an adjustment to a predetermined sequence of TMS stimulations for a subsequent sequence or set of sequences of TMS stimulations based on the determined extract measurements of local excitability and/or global connectivity.

17. A method in accordance with claim 16, wherein the determined extract measurements of local excitability includes amplitude, latency, signal power, area under curve properties or a combination thereof.

18. A method in accordance with claim 16, wherein the determined global connectivity includes interhemispheric conduction time and/or amplitude ration.

19. A method in accordance with claim 11, wherein the adjustment made to a subsequent sequence of TMS stimulations includes adjusting the intensity, duration, frequency and/or number of stimulation pulses, or a combination thereof.

20. A method in accordance with claim 11, further comprising the step of stimulating the subject at least once via a TMS coil during each wait period.

21. A method in accordance with claim 20, further comprising stimulating the subject less than 20 times, preferably less than 10 times and still more preferably between 3-7 times during each wait period.

22. (canceled)

23. A system comprising;
a Transcranial Magnetic Stimulation (TMS) coil device,
an electroencephalogram (EEG) device having at least one compensatory mechanism for preventing EEG saturations during stimulation from a TMS coil device,
a TMS device having a processor in electronic communication with a non-transitory computer readable medium, and a capacitor, said TMS device connected to the TMS coil and in communication with the EEG device.

24. (canceled)
